(19) United States
(12) Patent Application Publication
(10) Pub. No.: US 2010/0049043 A1
(43) Pub. Date: Feb. 25, 2010

(54) DOCKABLE ULTRASOUND SYSTEMS AND METHOD THEREOF

(75) Inventor: George Brock-Fisher, Andover, MA (US)

Correspondence Address: PHILIPS INTELLECTUAL PROPERTY & STANDARDS, P.O. BOX 3001, Briarcliff Manor, NY, 10510-8001, US

(73) Assignee: KONINKLIJKE PHILIPS ELECTRONICS N.V., Eindhoven (NL)

(21) Appl. No.: 12/515,977

(22) PCT Filed: Dec. 3, 2007
(86) PCT No.: PCT/IB2007/054906

§ 371 Date: May 22, 2009

Related U.S. Application Data
(60) Provisional application No. 60/868,382, filed on Dec. 4, 2006.

(51) Int. Cl. A61B 8/00

(52) U.S. Cl. 600/437

(57) ABSTRACT

A compact, portable ultra sound system is capable of docking with a larger, traditional cart-based ultrasound system. The docking can include a physical and an electrical connectivity between the compact portable ultrasound system and the larger traditional cart-based ultrasound system. The compact system is thus able to use the peripherals ofthe larger system and this provides for a more cost effective use of the larger system.

The present disclosure relates to a dockable ultrasound system. In particular the present disclosure relates to a compact ultrasound system that is portable and adapted to dock with a larger traditional cart-based ultrasound system and to share its peripherals and resources.

Many commercially available diagnostic ultrasound systems are available that provide premium performance. Typically these systems have a wide range of clinical applications and the best available imaging and application performance. These systems are large, usually weighing 300 pounds or more, being constructed onto a movable cart, to facilitate moving the system to different locations. These systems typically provide a large monitor for viewing images, peripheral devices such as video printers, VCRs, and different media recording devices (CD, DVR, MO disk). These systems also typically provide LAN connectivity to export images in DICOM or proprietary formats to offline review stations.

While provide the ultimate in diagnostic performance, they can not be easily transported to different wings of a hospital, or to outreach clinics. Therefore there is a need to develop premium performance ultrasound systems that are much lighter weight and that are much more easily transportable. However, these lightweight systems, by the nature of their design requirements, cannot easily incorporate all of the peripheral devices of the larger system and are therefore not a suitable replacement for these larger cart-based systems.

Since it is not possible for these compact ultrasound systems to provide all of the capabilities required in order to replace the larger, heavier systems completely, both types of systems are needed in the clinical environment.

It is therefore desirable to find a method and system for utilizing both the compact system and the larger cart-based system together in order to eliminate the need to move the larger system around the clinical facility and to provide the compact system with the use of the peripherals only available in the cart-based system.

The present invention provides a compact, portable ultrasound system that is capable of docking with a larger, traditional cart-based ultrasound system. The docking includes a physical and/or an electrical connectivity between the compact portable ultrasound system and the larger traditional cart-based ultrasound system. The compact system is thus able to use the peripherals of the larger system and this provides for a more cost effective use of the larger system.

Other objects will become readily apparent from the foregoing description and accompanying drawings in which:

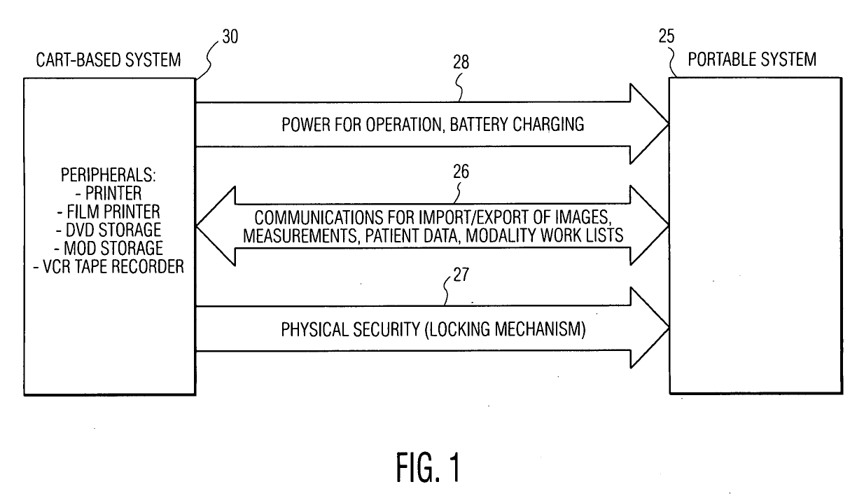

FIG. 1 is a block diagram of the present invention showing the dockable interface between the compact portable ultrasound system and the larger traditional cart-based ultrasound system; and

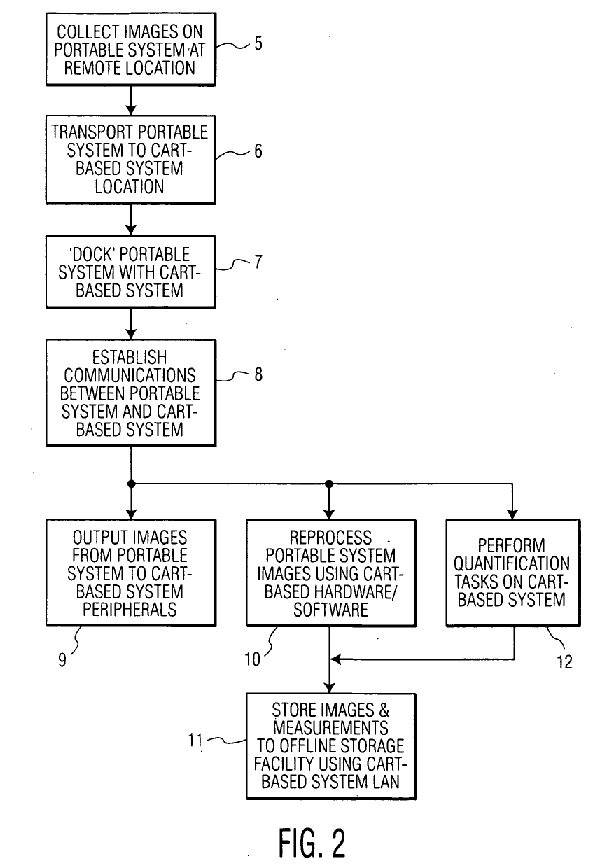

FIG. 2 is an operation flow chart of the present invention.

Referring now to the drawings, FIG. 1 is a block diagram of showing the docking between a portable compact ultrasound system and a larger traditional cart-based ultrasound system.

In FIG. 1 a communications port 26 connects the larger traditional ultrasound system 30 to the compact ultrasound system 25 in order to import/export images, measurements, patient data, and modality work lists. An electrical connectivity must be established in order to achieve docking between the two ultrasound systems. The electrical connectivity may be either a wired connection between the cart-based system and the portable system or a wireless connection. In addition there may be a physical interface between the cart-based system and the portable system.

The traditional cart-based system may be modified to include a ‘docking bay’ for the portable system. The cart-based system may be modified to provide a mechanical and an electrical docking bay into which the portable, compact system 25 may be placed. The portable system 25 includes physical and/or electrical features that enable it to dock or mate with the cart-based system. This physical connection is very similar to a laptop computer docking station.

A physical connection acts to secure the portable system to the cart-based system for the purpose of securing the portable system to the cart-based system both for support and for anti-theft security. The physical connection may be a manually operated locking device using a key or combination type lock; or alternatively the locking mechanism may be electronically operated from either system using a username and password to prevent unauthorized removal of the portable system from the cart-based system.

A physical security locking mechanism 27 safely secures and locks the larger ultrasound system to the compact ultrasound system when docked together. This may be either a manually operated locking device using a lock and a key or a combination type of lock or it may be electrically operated on either the larger or compact system by using a user name and password. The larger system 30 includes the following peripherals: a printer, a film printer, a DVD or MOD storage and a VCR tape recorder. The larger system 30 may be modified to include a docking bay for the compact portable system 25. The portable system 25 includes physical and/or electrical features that enable it to dock or mate with the larger system 30 to prevent unauthorized removal of the portable system 25 from the larger system 30. The docking bay of the cart-based system includes the following features:

Power may be supplied by the cart-based system to the portable system to enable the portable system to operate and/or charge its internal batteries An electrical interface connection, which may be a standard interface (i.e. LAN connection) or a custom interface which enables the computers in the cart-based system and the portable system to establish communications and transfer information between the two systems.

Specifically, the transfer of images, measurements, patient data, Modality Worklists, and HIPPA privacy/security information is included. The images or patient data previously collected on the portable system may be transmitted to the traditional system for storage, review or retransmission to an offline reading station.

Modality work lists may be downloaded from the traditional system, which have been previously received via its LAN connection, to the compact system for later use by a clinician.

Service patches may be transmitted to the compact system or diagnostic information may be transmitted from the compact system to the internet, following temporary storage on the traditional system.

A secure mechanical connection may be made which acts to prevent theft of the compact system. This mechanical connection may be activated/deactivated by a code entered on either machine.

The images from the compact system may be reviewed on the traditional system, taking advantage of the larger or higher resolution video monitor of the traditional system.

The aforementioned electrical data transfers may be accomplished by wireless communications between the cart-based system and the portable system such as but not limited to the 802.11 g which is a standard wireless protocol or else by any known wireless communications systems including other wireless router systems. It is understood that the invention is not limited to any specific wireless communications system.

Images which were collected on the compact system may be reprocessed to provide improved image quality or diagnostic information, using the greater computing capabilities of the traditional system.

In addition certain software modifications are required for the docking between the two ultrasound systems.

The cart-based system may be configured to add the docking bay. The cart-based system and the portable system through field upgrade or factory installation, will be programmed using computer software drivers and protocol have software which runs on it's computer(s) that is specially adapted to provide the foregoing applications:
Recognition of the portable system
Establishing of communications with the portable system
Authorization of security for patient privacy with the portable system
Transfer of images in both directions between the two systems
Transfer of measurements in both directions between the two systems
Transfer of Modality Worklists in both directions between the two systems
Transfer of software upgrades in both directions between the two systems
Transfer of system diagnostic information in both directions between the two systems
Control of reprocessing of images using the cart-based system software and/or hardware.
Control of directing specified images and data from the portable system to the cart-based system peripherals
Control of directing specified images and data from the portable system to an offline storage device via the cart-based networking connection (LAN, wireless).
Review of images collected on the portable system using the larger, higher resolution monitor of the cart-based system.
Activation/deactivation of a mechanical anti-theft security lock on the docking bay.

FIG. 2 is a flow chart showing the operation of the present invention.

First, ultrasound images are collected on a portable system 25 (in FIG. 2) as a remote location 5. The portable system 25 is then transported to the location of the larger cart-based traditional system 30 as seen in 6. The portable system optionally docks with the larger cart based system 7. Communications is established via the connection between the two systems 8. The communication between the two systems provides for the following:

The collected images 5 are transmitted from the portable system 25 to the peripherals of the larger traditional cart-based system 30 as shown in 9. These peripherals can include a monitor and image producing equipment. The collected images can be analyzed and/or reproduced using the larger cart-based system's hardware and software. 10 and then the images and measurements are stored to office storage facility using the cart-based system LAN 11.

Quantification tasks can also be performed on the collected images on the larger traditional cart-based ultrasound system 12. After quantification is performed the images and measurements can be stored to an offline storage facility using the large cart-based system's LAN.

While presently preferred embodiments have been described for purposes of the disclosure, numerous changes in the arrangement of method steps and apparatus parts can be made by those skilled in the art. Such changes are encompassed within the spirit of the invention as defined by the appended claims.

1. A compact ultrasound system which docks and operates with a larger ultrasound system, comprising:
a compact ultrasound system with stand-alone capability to image patients while being powered by a battery;
a larger cart-based ultrasound system with stand-alone capability to image patients, the larger ultrasound system including a monitor for viewing images, a plurality of peripheral devices located on the cart, and connectivity to export images over a network; and
the larger ultrasound system further comprises a docking bay for the compact ultrasound system so that the compact ultrasound system docks with the larger ultrasound system so that there is an electrical connection and an image data connection between the larger ultrasound system and the compact ultrasound system whereby images acquired by the compact ultrasound system can be transferred to the larger ultrasound system for processing by the larger ultrasound system, use of the peripheral devices with images acquired by the compact ultrasound system, and exporting compact ultrasound system images over the network.

2. The compact ultrasound system according to claim 1 wherein the image data connection is a wireless connection.

3. The compact ultrasound system according to claim 1 wherein the electrical connection is a wired connection.

4. The compact ultrasound system according to claim 1 wherein the docking bay provides a physical connection between the larger ultrasound system and the compact ultrasound system.

5. The compact ultrasound system according to claim 4 wherein the physical connection includes a security lock locking the compact system with the larger system.

6. The compact ultrasound system according to claim 1 wherein the electrical connection supplies power from the larger system to the compact ultrasound system to power the compact ultrasound system and to recharge the battery of the compact ultrasound system.

7. A system for utilizing a compact ultrasound system with a larger cart-based ultrasound system, comprising:
a battery-powered portable compact ultrasound system for imaging a patient without the use of the larger ultrasound system;
a larger cart-based ultrasound system for imaging a patient without the use of the compact ultrasound system;
the larger ultrasound system including a docking bay to dock with the compact ultrasound system to provide an electrical and image transfer connection between the larger ultrasound system and the compact ultrasound system.

8. The system according to claim 7 wherein said electrical connection is a wireless connection.

9. The system according to claim 7 wherein said electrical connection is a wired connection.

10. The system according to claim 7 wherein the docking bay provides a physical connection between the larger ultrasound system and the portable system.

11. The system according to claim 10 wherein said physical connection includes a security lock locking said compact system with said larger system.

12. The system according to claim 7 wherein the electrical connection supplies power from the larger system to the compact ultrasound system to power the compact system and to recharge a battery of the compact ultrasound system when the compact ultrasound system is docked in the docking bay.
